(19) United States
(12) Patent Application Publication
(10) Pub. No.: US 2006/0189965 A1
(43) Pub. Date: Aug. 24, 2006

(54) System,apparatus and method for large area tissue ablation

(76) Inventors: Emil Litvak, Yahud (IL); Dan V. Regelman, Haifa (IL); Boris Mezhericky, Haifa (IL)

Correspondence Address: Martin Moynihan;Prtsi Inc, PO Box 16446, Arlington, VA, 22215, US

(21) Appl. No.: 10/551,713

(22) PCT Filed: Mar. 29, 2004
(86) PCT No.: PCT/IL04/00285

§ 371 Date: Oct. 3, 2005

Related U.S. Application Data
(60) Provisional application No. 60/458,975, filed on Apr. 1, 2003.

(51) Int. Cl. A61B 18/18; A61C 3/00; A61B 19/00

(52) U.S. Cl. 606/10; 606/3; 606/13; 433/29; 128/898

(57) ABSTRACT

A method of ablating a material, the method comprising: (a) generating a beam of laser radiation in a form of plurality of pulses, the laser radiation having a wavelength suitable for ablating the material; and (b) within a duration of a pulse of the plurality of pulses, scanning the material by the beam, so as to transfer a predetermined amount of energy to each one of a plurality of locations of the material, the predetermined amount of energy being selected so as to ablate the material.

### FIELD AND BACKGROUND OF THE INVENTION

The present invention relates to tissue ablation and, more particularly, to tissue ablation using electromagnetic radiation, e.g., laser radiation. Most particularly, the present invention relates to hard tissue, such as teeth and bones, ablation using laser radiation. The invention present invention also relates to ablations of other materials such as ceramics.

Over the years, light and more specifically laser light has been used for the analysis, treatment, destruction or ablation of tissues.

The introduction of the laser technology in 1960, brought the light to a large variety of applications by producing spatially coherent light having very high intensity. Nowadays, laser technology has found many applications in medicine and biology, mostly in procedures which are related to the treatment of soft tissues.

Lasers are optical devices which produce intense and narrow beams of light at particular wavelengths by stimulating the atoms or molecules in a lasing material. Many types of lasing materials are known, including gases, liquids and solids. The lasers are typically named in accordance with the element or compound that lases when energized, such as carbon dioxide, argon, copper vapor, neodymium-doped yttrium-aluminum-garnet, erbium, holmium, ArF, XcCl, KrF, etc. When applied to human tissue, depending on wavelength selection and tissue combination, the beam of light produced by the laser is partially absorbed in a process which typically converts the light to heat. This is used to change the state of the tissue for purposes of etching or cutting via tissue destruction or ablation.

Laser destruction or ablation of unwanted soft tissue is widely achieved, either through a direct interaction between the electromagnetic field of the laser beam and the tissue, or through activation of photochemical reactions using light-activated molecules which are injected into or otherwise administered to the tissue prior to laser radiation, a procedure known as photodynamic therapy (PDT).

Unwanted hard tissues, such as dental enamel and dentin are traditionally removed by mechanical means, such as drills, etc. Such procedures are extremely uncomfortable, painful to the patient, and produce results having quite a few drawbacks.

Laser use for dental enamel surgery was reported as early as 1964 using a ruby laser. However, lasers have not generally been used clinically until early 90's for surgical processes, including tooth drilling, because of the large amount of damage to nearby tissue that was often associated with such laser drilling.

The use of laser radiation in dental procedures is attractive because such radiation can be focused to a very small area and is thus compatible with the dimensional scale of the oral cavity. Additional advantages of laser based dental procedures include minimal or no need for anesthetic, minimal or no pain, minimal discomfort, minimal chair time, no drill sound, minimal or no bleeding, fast healing and reduced chances of postoperative infections and complications.

While a number of devices for dental treatment of this type have been proposed, these devices have not proven to be of practical use notably because even the most effective of these devices are useful only under limited and very precisely defined conditions. Pulsed lasers and lasers producing infrared radiation have been developed both for soft tissue and bone ablation, and, although were found to be less damaging than other lasers, they still yielded unsatisfactory results.

U.S. Pat. No. 4,818,230, the content of which is hereby incorporated by reference, discloses a method of removing decay from teeth using a yttrium-aluminum-garnet (YAG) laser doped with Nd⁺³. The YAG laser was used to eradicate tooth decay located in the dentin without significantly heating the tooth and thus without damaging the nerve. YAG laser has also been used to remove incipient carious lesions and/or stain from teeth (U.S. Pat. No. 4,521,194). This use of a YAG laser was found to slightly fuse the crystals which form the tooth enamel and make the tooth enamel more impervious to decay.

A variant of the Nd⁺³:YAG laser employs YAG doped with Erbium (Er), which is a metallic element of the rare-earth group that occurs with yttrium and was found to be useful as a source of laser irradiation. This variant is known as Er:YAG laser. The Er:YAG laser is a solid-state pulsed laser which has a maximum emission in the mid-infrared region at 2.94 μm or 2.79 μm.

FIG. 1 shows approximate absorption curves of several tissue components. As can be seen from FIG. 1, the absorption coefficient of water acquires a sharp peak at a wavelength of 2.94 μm, where the Er:YAG laser produces its maximal power. For example, the water absorption coefficient for radiation produced by an Er:YAG laser is about ten times that of radiation produced by a CO₂ laser.

The dynamic of hard tissue ablation using Er:YAG laser is apparently as follows [J. A. Izatt et al., IEEE J. Quantum Electron, 26:2261, 1990; J. T. Walsh and T. F. Deutsch, Appl. Phys., B52:217, (1991); R. Hibs and U. Keller, SPIE Proc., 1880:156 (1993)]: Er:YAG laser results in water in the target tissue absorbing the radiant energy and heating to boiling to produce water vapor. The water vapor builds up in pressure at the irradiated site until a micro-explosion occurs and the surrounding hydroxyapatite crystal is ablated.

It is generally known that, using a pulsed laser system, the individual pulses of the laser radiation may exceed the threshold of critical energy concentration (which varies by material), so that biological material can be removed without creating a significantly increased temperature in the areas peripheral to the location of treatment. To achieve this result, however, extremely short laser pulses (on the order of nanoseconds) must be used, and the thickness of the biological material removed by this method is between 10 and 50 μm. To reach worthwhile rates of material removal, given such a tiny thickness per individual laser pulse, it is necessary to increase the repetition rate of the laser pulses. Because hard biological material has a limited heat transfer capacity, however, increasing the repetition rate of the pulses rapidly leads to an accumulation of heat around the zone of removal, and hence leads to thermal damage of the areas peripheral to the treatment area. For example, it is known that a tooth may bare a temperature inclement of no more that 5° C., without undergoing irreversible damage. To reduce the level of laser induced thermal damage, various types of cooling equipment are used, which introduce a continuous jet of water or a continuous flow of air onto the treatment site.

A known problem with ablation of hard tissues is that the ablation process saturates after few tens of pulses. This fact is explained [B. Majaron et al., Appl. Phys. B66:479 (1998); J. T. Walsh and T. F. Deutsch, Lasers Surg. Med. 9:327 (1989); G. B. Altshuler et al., Proc. SPIE 2080:10 (1993)] by excessive heating of the tissue under the ablated region, which causes evaporation of water from the tissue. At this stage, the ablation process is stalled and can be continued only by the external application of water. The water hits the tissue surface, wets it and probably penetrates into the upper layers of the tissue itself, thereby ensures the continuation of the micro-explosions. Any absorption of laser radiation by the tissue other than the tissue which is to be ablated causes the undesired effect of saturation.

An additional effect that strongly reduces the ablation efficiency is the so called, debris screening. Since debris removal is a mechanical process, its time scale is relatively long, compared to the pulse duration. While ejected from the irradiated area, the debris from a screening cloud, which typically accommodates the space between the laser source and the tissue. Thus, a significant amount of the laser energy is absorbed by tissue which has already been ablated. This effect causes a dramatic reduction in the ablation efficiency and becomes even more significant with the increase of the laser pulse duration or the laser pulse energy.

The above problems associated with the process of hard tissue laser ablation have only been partially solved by the presently known technologies. It particular, the excessive heating has been only partially overcome, by selecting short pulse duration and small beam areas (of the order of 1 mm).

There are some procedures, however, that particularly require removal of large area of dental tissue or bone. In these procedures, nowadays, due to the above identified problems, mechanical drill is typically used. For example, during a preparation of a tooth to be crowned, the dentist uses 4 to 7 drills, in several depths and widths to complete the preparation. Each such drill change requires halting the operation for 15-30 seconds, thus considerably increasing the overall operation time and thereby the discomfort to the patient. In addition, the drilling process is extremely long and uncomfortable.

Many attempts have been made to design laser systems for the purpose of ablating hard tissues, such as teeth and bones.

U.S. Pat. No. 5,636,983 describes a laser cutting apparatus in which the pulse parameters (such as the pulse duration or time intervals between the pulses) are adjustable. In addition to the cooling water spray, U.S. Pat. No. 5,636,983 uses a polishing member which is applied in a certain time sequence, together with the water spray, air and the laser energy. The addition of a polishing member improves the performance of the laser cutting apparatus by means of removing a carbonized layer on dentin, disabling the formation of a fused layer on dentin, making the margins of the irradiated area more regular and avoiding cracking and damaging of dental pulp due to temperature rise.

U.S. Pat. No. 6,086,366 describes a device for hard tissue ablation in which a laser beam is directed on the tissue. The device includes also a distance measurement device which monitors the depth of material removal, so that while the material is being removed, the depth of material removal is measured by means of another laser. The laser beam, according to U.S. Pat. No. 6,086,366, may be relocated such that successive ablation points lie as far apart as possible within the area to be machined for permitting an interim cooling of the previously-irradiated ablation region, thus attaining a homogeneous heat distribution. This technique, however, fail to provide a solution to the problem of over-heating of the irradiated spot due to long pulse duration.

U.S. Pat. Nos. 6,156,030 and 6,482,199 disclose optimization procedures for the laser parameters (pulse energy, pulse duration, intervals between successive pulses) so as to minimize the damage to the underlying and surrounding tissues. Specifically, the optimization is directed at removing the residual energy so as to minimize collateral thermal damage. The optimization, however, refers to the ablation with a laser spot which does not spatially move in time.

Additional possibilities for the application of lasers to the field of dentistry in particular, and to hard tissue ablation in general, have been proposed (see, e.g., U.S. Pat. Nos. 5,785,703, 5,435,724 and 5,968,035) by the use of lasers that emit high intensity pulses of ultraviolet light. Theses lasers typically use nanosecond range pulse durations which contribute to defining a different regime of laser-tissue interaction. Short wavelength ultraviolet photons are energetic enough to directly break chemical bonds in organic molecules. Although ultraviolet lasers vaporize a material target with minimal thermal energy transfer to adjacent tissue, such lasers suffer from a major limitation due to a relatively small active area, which prohibits the use of ultraviolet lasers for ablating large areas of hard tissues.

Other prior art documents of interest include U.S. Pat. Nos. 5,720,894, 5,415,652, 5,458,594, 5,303,026, 5,267,856, 5,501,599, 5,762,501, 5,885,082, 5,342,198 and 6,451,009; and U.S. Application Nos. 2002/0183724, 2001/0009250, 2002/0169379 and 2002/0021511.

None of the above documents, however, provide adequate solutions to the problems associated with overheating of the irradiated area due to long pulse duration and/or ablation of large areas of hard tissues.

There is thus a widely recognized need for, and it would be highly advantageous to have, a system, apparatus and method for hard tissue ablation devoid of the above limitations.

### SUMMARY OF THE INVENTION

According to one aspect of the present invention there is provided a method of ablating a material, the method comprising: (a) generating a beam of laser radiation in a form of plurality of pulses, the laser radiation having a wavelength suitable for ablating the material; and (b) within a duration of a pulse of the plurality of pulses, scanning the material by the beam, so as to transfer a predetermined amount of energy to each one of a plurality of locations of the material, the predetermined amount of energy being selected so as to ablate the material.

According to further features in preferred embodiments of the invention described below, the at least one scanning-parameter, the duration of the pulse and the predetermined amount of energy are selected to perform a dental procedure.

According to still further features in the described preferred embodiments the dental procedure is selected from the group consisting of crown preparation, dental implantation, caries removal, endodontic treatment, bones surgery, enamel and dentin preparation and conditioning.

According to another aspect of the present invention there is provided a method of crowning a tooth, the method comprising: (a) generating a beam of laser radiation in a form of plurality of pulses, the laser radiation having a wavelength suitable for ablating the tooth; (b) within a duration of a pulse of the plurality of pulses, scanning the tooth by the beam, so as to transfer a predetermined amount of energy to each one of a plurality of locations of the tooth, the predetermined amount of energy being selected so as to ablate the tooth; (c) repeating the step (b) a number of times which is required to ablate an external surface of the tooth, thereby revealing a reduced surface of the tooth; and (d) providing a crown having an inner surface geometrically compatible with the reduced surface of the tooth, and attaching the crown onto the tooth.

According to an additional aspect of the present invention there is provided a method of treating a tumor in a bone, the method comprising: (a) generating a beam of laser radiation in a form of plurality of pulses, the laser radiation having a wavelength suitable for ablating the bone; and (b) within a duration of a pulse of the plurality of pulses, scanning the bone by the beam, so as to transfer a predetermined amount of energy to each one of a plurality of locations of the bone, the predetermined amount of energy being selected so as to ablate the tumor.

According to further features in preferred embodiments of the invention described below, the scanning is characterized by at least one scanning-parameter, the at least one scanning-parameter is selected from the group consisting of a scanning-frequency, a scanning-velocity, a scanning-acceleration, a scanning-amplitude, a scanning-angle, a scanning-pattern and a scanning-duration.

According to still further features in the described preferred embodiments the scanning is selected from the group consisting of a one-dimensional scanning, a two-dimensional scanning and a three-dimensional scanning.

According to still further features in the described preferred embodiments the at least one scanning-parameter is selected so as to minimize heating of internal layers of the material.

According to still further features in the described preferred embodiments the at least one scanning-parameter is selected so as to minimize shifts in an absorption curve of at least one component present in the material.

According to still further features in the described preferred embodiments the at least one scanning-parameter is selected so as to allow ablation of substantially large areas of the material.

According to still further features in the described preferred embodiments the laser radiation has a power sufficient for ablation of substantially large areas of the material.

According to still further features in the described preferred embodiments the duration of the pulse is selected so as to allow ablation of substantially large areas of the material.

According to still further features in the described preferred embodiments the at least one scanning-parameter is selected so as to provide a predetermined ablation pattern.

According to still further features in the described preferred embodiments the predetermined ablation pattern is selected from the group consisting of a repetitive pattern, a cylindrical pattern and an irregular pattern.

According to still further features in the described preferred embodiments the compensating the transient non-uniformities of the intensity distribution is by selecting the scanning-velocity inversely proportional to the intensity distribution.

According to still further features in the described preferred embodiments the at least one scanning-parameter is selected so as to compensate flux non-uniformities caused by different impinging angles of the beam on the plurality of locations of the material.

According to still further features in the described preferred embodiments the compensating the flux non-uniformities is by selecting the scanning-velocity to be small for large impinging angles and large for small impinging angles, the large impinging angles and the small impinging angles being measured relative to an imaginary line positioned normal to the material.

According to still further features in the described preferred embodiments the scanning is by dynamically diverting the beam, so as to provide a substantially constant impinging angle of the beam on each of the plurality of locations of the material.

According to still further features in the described preferred embodiments the method further comprises cooling the material during the scanning.

According to still further features in the described preferred embodiments the method further comprises continuously determining at least one impinging-parameter of the beam on the material.

According to still further features in the described preferred embodiments the continuously determining the at least one impinging parameter is by an additional laser beam.

According to still further features in the described preferred embodiments the additional laser beam is characterized by a wavelength selected so as not to damage the material.

According to still further features in the described preferred embodiments the method further comprises terminating the laser radiation if the at least one impinging-parameter is in a predetermined risk range.

According to still further features in the described preferred embodiments the method further comprises focusing the beam on a surface of the material using at least one focusing element.

According to still further features in the described preferred embodiments the at least one scanning-parameter is selected so as to minimize debris screening.

According to still further features in the described preferred embodiments the at least one scanning-parameter is selected so as to compensate spatial non-uniformities of intensity distribution of the laser radiation.

According to still further features in the described preferred embodiments the compensating the spatial non-uniformities of the intensity distribution is by rotating the beam about a longitudinal axis.

According to still further features in the described preferred embodiments the compensating the spatial non-uniformities of the intensity distribution is by positioning an optical element in a light-path of the beam and rotating the optical element about a longitudinal axis.

According to still further features in the described preferred embodiments the compensating the spatial non-uniformities of the intensity distribution is by positioning a passive beam homogenizer in the light path of the beam.

According to still further features in the described preferred embodiments the at least one scanning-parameter is selected so as to compensate transient non-uniformities of intensity distribution of the laser radiation within the duration of the pulse.

According to still further features in the described preferred embodiments the compensating the transient non-uniformities of the intensity distribution is by selecting the scanning-velocity to be inversely proportional to the intensity distribution.

According to yet another aspect of the present invention there is provided an apparatus for scanning a material by a beam of laser radiation being in a form of plurality of pulses, the apparatus comprising a scanning assembly for dynamically diverting the beam, within a duration of a pulse of the plurality of pulses, so as to transfer a predetermined amount of energy to each one of a plurality of locations of the material, thereby to scan the material by the beam.

According to further features in preferred embodiments of the invention described below, the apparatus further comprises a synchronizer for synchronizing the scanning assembly and a laser device generating the beam.

According to still further features in the described preferred embodiments the scanning assembly is designed and constructed to scan substantially large areas of the material.

According to still further features in the described preferred embodiments the scanning assembly is designed and constructed to generate a predetermined scanning pattern.

According to still further features in the described preferred embodiments the apparatus further comprises an optical element positioned in a light-path of the beam and operable to rotate about a longitudinal axis so that the beam is rotated about the longitudinal axis, hence compensating the spatial non-uniformities of the intensity distribution.

According to still further features in the described preferred embodiments the apparatus further comprises an arm interface for mounting the scanning assembly to an articulated arm. According to still further features in the described preferred embodiments the apparatus further comprises a handpiece, hingedly attached to the scanning assembly and operable to rotate to a plurality of open positions, the handpiece being capable of guiding the beam therethrough in each one of the plurality of open positions.

According to still further features in the described preferred embodiments the apparatus further comprises a light collector for collecting the additional laser beam when the additional laser beam is reflected from the material, thereby to determine at least one impinging-parameter of the beam on the material.

According to still further features in the described preferred embodiments the apparatus further comprises at least one waveguide and an additional synchronizer communicating with the laser device, the at least one waveguide being designed and constructed for directing the additional laser beam to the additional synchronizer, and the additional synchronizer being designed and constructed to synchronize the laser device and the additional laser beam.

According to still another aspect of the present invention there is provided a system for ablating a material, the system comprising: (a) a laser device for generating a beam of laser radiation in a form of plurality of pulses, the laser radiation having a wavelength suitable for ablating the material; and (b) a scanning assembly, electrically communicating with the laser device, the scanning assembly being capable of scanning the material by the beam, within a duration of a pulse of the plurality of pulses, so as to transfer a predetermined amount of energy to each one of a plurality of locations of the material, the predetermined amount of energy being selected so as to ablate the material.

According to further features in preferred embodiments of the invention described below, the scanning assembly comprises a synchronizer for synchronizing the scanning assembly and the laser device.

According to still further features in the described preferred embodiments the synchronizer is selected from the group consisting of an optical synchronizer and an electrical synchronizer.

According to still further features in the described preferred embodiments the scanning assembly is operable to dynamically divert the beam thereby to scan the material by the beam.

According to still further features in the described preferred embodiments the scanning assembly comprises at least one optical element positioned in a light-path of the beam, the at least one optical element being operable to rotate thereby to dynamically divert the beam.

According to still further features in the described preferred embodiments the scanning assembly is operable to preserve a substantially constant impinging angle of the beam on each of the plurality of locations of the material.

According to still further features in the described preferred embodiments the scanning assembly is operable to generate scanning which is selected from the group consisting of a one-dimensional scanning, a two-dimensional scanning and a three-dimensional scanning.

According to still further features in the described preferred embodiments the scanning assembly is designed and constructed to scan the material in such a manner that heating of internal layers of the material is minimized.

According to still further features in the described preferred embodiments the scanning assembly is designed and constructed to scan the material in such a manner that debris screening is minimized.

According to still further features in the described preferred embodiments the scanning assembly is designed and constructed to scan the material in such a manner that shifts in an absorption curve of at least one component present in the material are minimized.

According to still further features in the described preferred embodiments the scanning assembly is designed and constructed to scan the material in such a manner that substantially large areas of the material are ablated.

According to still further features in the described preferred embodiments the laser device is designed and constructed to generate laser radiation having a power sufficient for ablation of substantially large areas of the material.

According to still further features in the described preferred embodiments the laser device is designed and constructed so that the duration of the pulse is sufficient for allowing ablation of substantially large areas of the material.

According to still further features in the described preferred embodiments the scanning assembly is designed and constructed to generate a predetermined ablation pattern.

According to still further features in the described preferred embodiments the predetermined ablation pattern is selected from the group consisting of a repetitive pattern, a cylindrical pattern and an irregular pattern.

According to still further features in the described preferred embodiments the scanning assembly is designed and constructed to scan the material in such a manner that spatial non-uniformities of intensity distribution of the laser radiation are compensated.

According to still further features in the described preferred embodiments the scanning assembly comprises an optical element positioned in a light-path of the beam and operable to rotate about a longitudinal axis so that the beam is rotated about the longitudinal axis, hence compensating the spatial non-uniformities of the intensity distribution.

According to still further features in the described preferred embodiments the scanning assembly comprises a passive beam homogenizer positioned in a light-path of the beam and operable to compensate the spatial non-uniformities of the intensity distribution.

According to still further features in the described preferred embodiments the scanning assembly is designed and constructed to scan the material in such a manner that transient non-uniformities of intensity distribution of the laser radiation within the duration of the pulse are compensated.

According to still further features in the described preferred embodiments the scanning assembly is operable to provide a scanning-velocity which is inversely proportional to the intensity distribution, thereby to compensate the transient non-uniformities of the intensity distribution.

According to still further features in the described preferred embodiments the scanning assembly is designed and constructed to scan the material in such a manner that flux non-uniformities, caused by different impinging angles of the beam on the plurality of locations of the material, are compensated.

According to still further features in the described preferred embodiments the scanning assembly is operable to provide a small scanning-velocity for large impinging angles and a large scanning-velocity for small impinging angles, thereby to compensate the flux non-uniformities, the large impinging angles and the small impinging angles being measured relative to a normal to the material.

According to still further features in the described preferred embodiments the system further comprises at least one articulated arm onto which the scanning assembly is mounted, the at least one articulated arm and the scanning assembly are constructed and designed to operate within or adjacent to an oral cavity.

According to still further features in the described preferred embodiments the system further comprises a handpiece, hingedly attached to the scanning assembly and operable to rotate to a plurality of open positions, the handpiece being capable of guiding the beam therethrough in each one of the plurality of open positions.

According to still further features in the described preferred embodiments the system further comprises a user interface device electrically communicating with the scanning assembly and capable of transmitting scanning-parameters to the scanning assembly.

According to still further features in the described preferred embodiments the laser device is selected from the group consisting of an Er based laser device, a Ho:YAG laser device, a carbon-dioxide laser device, an Nd based laser device and a laser diode device.

According to still further features in the described preferred embodiments the Er based laser device is selected from the group consisting of an Er:YAG laser device, Er:YSGG laser device and Er:glass laser device.

According to still further features in the described preferred embodiments the Nd based laser device is selected from the group consisting of an Nd:YAG laser device an Nd:YLF laser device and an Nd:glass laser device.

According to still further features in the described preferred embodiments the laser radiation is polarized.

According to still further features in the described preferred embodiments the wavelength is in an infrared scale.

According to still further features in the described preferred embodiments the wavelength is in an ultraviolet scale.

According to still further features in the described preferred embodiments the wavelength is in a visible scale.

According to still further features in the described preferred embodiments the wavelength is a characteristic wavelength of an absorption curve of water.

According to still further features in the described preferred embodiments the wavelength is about 2.94 micrometers.

According to still further features in the described preferred embodiments the system further comprises a cooling apparatus.

According to still further features in the described preferred embodiments the cooling apparatus is a liquid sprayer.

According to still further features in the described preferred embodiments the liquid is water.

According to still further features in the described preferred embodiments the liquid is airflow.

According to still further features in the described preferred embodiments the system further comprises a mechanism for continuously determining at least one impinging-parameter of the beam on the material.

According to still further features in the described preferred embodiments the at least one impinging-parameter is selected from the group consisting of an impinging-location and an impinging-angle.

According to still further features in the described preferred embodiments the mechanism for the continuously determining the at least one impinging-parameter is an additional laser device, operable to generate an additional laser beam.

According to still further features in the described preferred embodiments the system further comprises a light collector for collecting the additional laser beam when the additional laser beam is reflected from the material, thereby to determine at least one impinging-parameter of the beam on the material.

According to still further features in the described preferred embodiments the system further comprises at least one waveguide and an additional synchronizer communicating with the laser device, the at least one waveguide being designed and constructed for directing the additional laser beam to the additional synchronizer, and the additional synchronizer being designed and constructed to synchronize the laser device and the additional laser beam.

According to still further features in the described preferred embodiments the material is a hard material.

According to still further features in the described preferred embodiments the material is a hard tissue.

According to still further features in the described preferred embodiments the material is selected from the group consisting of enamel, dentin and bone tissue.

According to still further features in the described preferred embodiments the material forms a part of a tooth of a human.

According to still further features in the described preferred embodiments the material forms a part of a tooth of an animal.

According to still further features in the described preferred embodiments the material is a tooth.

According to still further features in the described preferred embodiments the optical element is selected from the group consisting of a lens, a mirror and a prism.

The present invention successfully addresses the shortcomings of the presently known configurations by providing a system, apparatus and method for hard tissue ablation, which enjoy properties far exceeding prior art.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

Implementation of the method and system of the present invention involves performing or completing selected tasks or steps manually, automatically, or a combination thereof. Moreover, according to actual instrumentation and equipment of preferred embodiments of the method and system of the present invention, several selected steps could be implemented by hardware or by software on any operating system of any firmware or a combination thereof. For example, as hardware, selected steps of the invention could be implemented as a chip or a circuit. As software, selected steps of the invention could be implemented as a plurality of software instructions being executed by a computer using any suitable operating system. In any case, selected steps of the method and system of the invention could be described as being performed by a data processor, such as a computing platform for executing a plurality of instructions.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 shows approximate absorption curves of several tissue components;

FIG. 2 is a flowchart of a method of ablating a material, according to one aspect of the present invention;

FIG. 3 is a graph showing the energy absorbed by water as a function of the wavelength;

FIG. 4a shows the shape of a 400 microseconds pulse in the time-intensity plane;

FIG. 4b shows the energy absorbed in a material as a function of time for the pulse duration of FIG. 4a;

FIG. 5 is a schematic illustration of an apparatus for scanning a material by a beam of laser radiation, showing also a light-path of the beam of laser radiation, according to another aspect of the present invention;

FIGS. 6a-b are schematic illustrations of a scanning assembly, according to a preferred embodiment of the present invention;

FIGS. 7a-c is schematic illustration of a handpiece of the apparatus, according to a preferred embodiment of the present invention;

FIG. 8 is a simplified illustration of a light-path of an additional laser beam according to a preferred embodiment of the present invention;

FIGS. 9a-b are schematic illustrations of a configuration which may be used for terminating and reactivating the laser beam, according to a preferred embodiment of the present invention;

FIG. 10 is a schematic illustration of a system for ablating a material, according to an additional aspect of the present invention;

FIG. 11 is a flowchart of a method of crowning a tooth, according to yet another aspect of the present invention;

FIG. 12 shows results of measurements of an absorption coefficient of water as a function of the applied energy density;

FIG. 13 shows results of depth profiles of the laser intensity distribution at various times during a laser pulse having a total energy of 1000 mJ per pulse and a laser spot diameter of 0.3 mm;

FIG. 14 shows the total amount of the absorbed energy within the top 40 μm of tissue for a pulse of 1000 mJ applied to different spot sizes;

FIG. 15 is a schematic illustration of an experimental system for ablating hard tissues;

FIG. 16 is a series of 10 images of a tooth taken at different times during a laser pulse;

FIG. 17 is a schematic illustration of an experimental system for ablating hard tissues, which include a scanning-assembly and a scanning control unit;

FIG. 18a is an image of irradiated enamel for different scanning-frequencies;

FIG. 18b is a graph showing the width of the formed groove as a function of the scanning-frequency;

FIG. 18c is an image of enamel scanned at a scanning-frequency of 1150 Hz;

FIG. 18d is an image of enamel scanned at a scanning-frequency of 35 Hz;

FIG. 19a shows the pulse shape and the position of the laser spot as a function of time within the duration of the laser pulse, for a constant scanning-velocity;

FIG. 19b illustrates the amount of energy delivered to each location on the enamel sample, when the constant scanning-velocity was used;

FIG. 19c shows the pulse shape and a profile of a modulated scanning-velocity which was used for compensating the effect of transient non-uniformities;

FIG. 19d shows several positions of the laser spot on the enamel, when the modulated scanning-velocity was employed;

FIGS. 20a-b are images of enamel after a 90 seconds ablation procedure;

FIG. 21 is an image of dentine after a 30 seconds ablation procedure; and

FIGS. 22a-b are images of a bone tissue after a 30 seconds ablation procedure.

### DESCRIPTION OF THE PREFERRED EMBODIMENTS

The present invention is of a system, apparatus and method for ablation, which can be used for non-mechanical ablation using electromagnetic radiation, laser radiation in particular. Specifically, the present invention can be used to ablate large areas of a hard tissue, such as, but not limited to, enamel, dentin and bone tissue, or other hard materials such as, but not limited to, ceramic and earthenware materials. For example, the present invention can be used for performing dental procedure, such as, but not limited to, crowning of a tooth, in humans or animals.

The principles and operation of a system, apparatus and method for ablating a material according to the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

According to one aspect of the present invention there is provided a method of ablating a material. The material may be any material (hard or soft) suitable for being ablated by laser radiation, such as a tissue. For example, the material may be a part of a tooth (e.g., dentin or enamel) or a part of a bone of an animal, such as a mammal. Thus, the method of the present invention can be utilized in numerous procedures, such as, but not limited to, dental procedures, orthopedic procedures (e.g., bone transplantations), bone tumor (metastatic tumor) treatments and veterinary procedure.

For example, as further detailed hereinunder, the method is preferably employed in a dental operation, such as, but not limited to, crowning of a tooth. One advantage of the method of the present embodiment over prior art dental procedures is that the procedure may be exploited for tooth ablation in a predetermined geometrical surface.

According to a preferred embodiment of the present invention the method can also be employed in the field of bones surgery. The advantage of this embodiment is that laser ablation is extremely effective for ablating large metastatic bone tumors for the purpose of destroying the tumor and for the purpose of relieving pain associated with such metastases. Thus, the method is preferably used for ablating metastatic tumors, for the purpose of reducing the volume of the metastatic tumor, killing the entire tumor volume, or at least a portion thereof, and/or for the alleviation of pain for the patient.

In other embodiments of the present invention the method can be used for ablating a material other than a tissue, e.g., ceramics and the like.

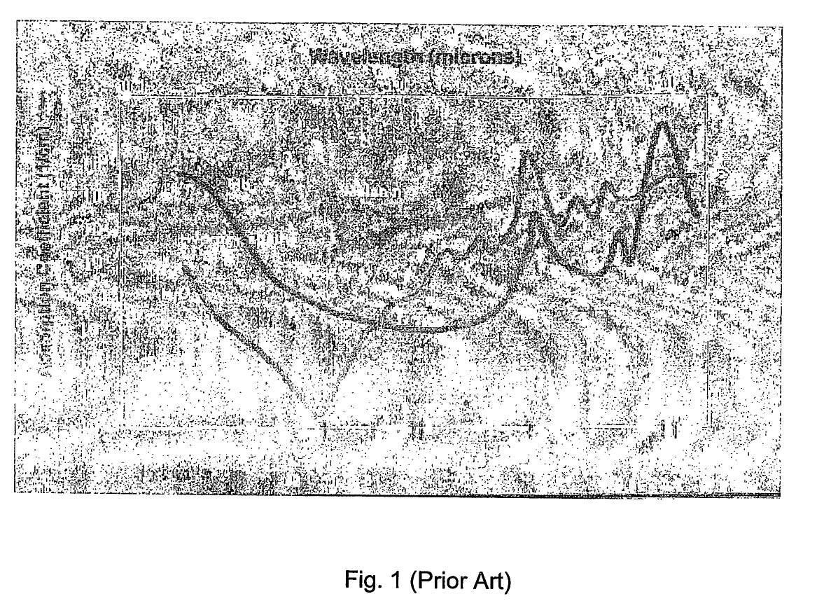

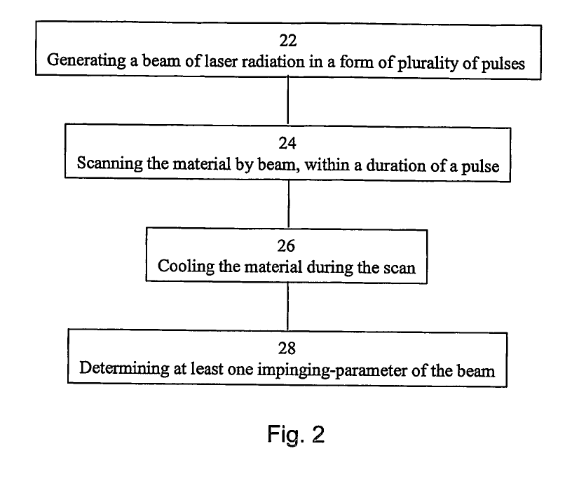

Referring now to the drawings, the method comprises the following method steps which are illustrated in the flowchart of FIG. 2. Hence, in a first step, designated by Block 22, a beam of laser radiation, having a wavelength suitable for ablating the material, is generated in a form of plurality of pulses. The wavelength may be any wavelength which can ablate the material, either via photo-acoustic effects or by directly breaking chemical bonds in the material. Thus, the wavelength may be in an infrared, ultraviolet or visible scale. For example, for ablating the material via photo-acoustic effects the wavelength may be a characteristic wavelength of an absorption curve of a component of the material, e.g., water, for which the absorption curve has a sharp peak at about 2.94 μm (see FIG. 1). Thus, a preferred, but not limited wavelength of the laser radiation is about 2.94 μm.

In a second step of the method, designated in FIG. 2 by Block 24, the material is scanned by the beam, within a duration of a pulse of the plurality of pulses. The scanning is performed in such a manner that a predetermined amount of energy is transferred to each one of a plurality of locations of the material, where the predetermined amount of energy is selected so as to ablate the material.

According to a preferred embodiment of the present invention the power of the laser radiation and the pulse duration are sufficient for ablating substantially large areas of the material, preferably above 1 mm², more preferably above 5 mm², most preferably above 10 mm². As further detailed hereinbelow and exemplified in the Examples section that follows, the scanning procedure allows the power of the laser radiation to be considerably higher than the laser radiation used by prior art techniques. A preferred range of laser pulse energy is from about 0.5 Joule to about 10 Joules. It is to be understood, however, that in other applications the range can significantly vary, depending on the beam spot size, the ablated material, the processed area, the laser type, etc.

The pulse duration is selected so that the beam covers a plurality of locations within the pulse duration, were the dimension of each location is approximately equal to the cross-sectional area of the beam. For example, for effective scanning the pulse duration may be larger than the diameter of the cross-sectional area of the beam divided by the scanning-velocity.

Beside scanning-velocity, other parameters which characterize the scanning may predetermined for the purpose of optimizing the procedure. These parameters include, but are not limited to, scanning-frequency, scanning-acceleration, scanning-amplitude, scanning-angle, scanning-pattern and scanning-duration. One or more of these scanning-parameters is preferably selected so as to allow ablation of substantially large areas of the material.

According to a preferred embodiment of the present invention the scanning may be realized in many ways, depending on the application for which the invention is used. For example, the scanning may be along a curve or a straight line (one-dimensional scanning), so as to ablate the material along the curve or a straight line. Alternatively, the scanning may be along a plurality of intersecting or parallel curves or straight lines (two-dimensional scanning) so as to ablate a pre-selected area of the material. Still alternatively, the scanning may also be a combination of a one- and two-dimensional scanning so as to ablate a pre-selected volume of the material (three-dimensional scanning).

As stated in the Background section above, for ablating a material in general, and a biological material in particular, without a significant change in the temperature of the material, extremely short laser pulses (on the order of nanoseconds) have been used in prior art techniques. Such short pulses unavoidably significantly increase the time required to ablate the material along the desired pattern (curve, area, volume), in particular when the desired pattern is large compared to the cross-sectional area of the beam.

A particular feature of the present invention is the scanning procedure, which is executed within the duration of a single pulse, and may be repeated for more than one pulse (e.g., for each pulse). The scanning procedure allows the use of larger pulse duration by a judicious selection of one or more scanning-parameters, as further explained hereinunder.

Hence, while the material is scanned by the beam, the amount of energy carried by a single pulse is distributed among the plurality of locations of the material covered by the beam spot. Thus, a particular (spot-sized) location absorbs an amount of energy which is smaller than the amount of energy which would have been absorbed had the pulse impinged on the particular location. For example, if one or more of the scanning parameters are set so that within a single pulse n locations are covered, then, the duration of the pulse may be n times larger compared to prior art techniques.

The present invention successfully addresses the problem of saturation in the ablation process, which is caused by several phenomena. One phenomenon, typically occurring when the ablating is governed by micro-explosives of water molecules present in the material, is the excessive heating of the internal layers of the material under the ablated region. Such excessive heating cause the evaporation of water from the material hence reduces the efficiency of the ablation process.

Another phenomenon is related to the dynamical behavior of the absorption curve of the material. When the material absorbs energy, the profile of its absorption curve changes, for example, due to energy-dependent inter-molecular interactions. Once the absorption coefficient changes, the efficiency of the ablation process, being characterized by a well-defined and constant wavelength, is reduced.

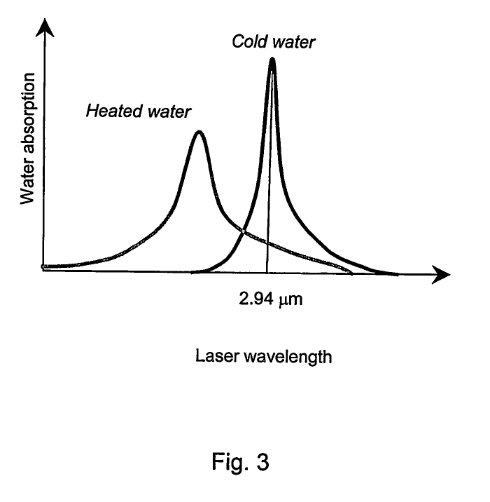

Reference is now made to FIG. 3, which is a graph showing the energy absorbed by water as a function of the wavelength near a wavelength of 2.94 μm. The sharp peak observed at a wavelength of 2.94 μm for cold water is shifted to a lower wavelength for heated water.

According to a preferred embodiment of the present invention the scanning is done so that each location of the material is irradiated substantially while the absorption coefficient is optimal.

Thus, one or more scanning-parameters are preferably selected so as to minimize the effects of at least one of the above phenomena. Specifically, according to a preferred embodiment of the present invention scanning-parameters are selected so as to minimize (i) heating of internal layers of the material and (ii) shifts in an absorption curve of at least one component present in the material. These two minimizations are not conflicting and therefore may be achieved simultaneously, for example, by selecting the exposure time of each location to be sufficiently long for ablating the material, yet not longer than the irradiation time during which the absorption coefficient is optimal, or not longer than the irradiation time required to initiate water micro explosion from the material.

An additional phenomenon which significantly reduces the efficiency of the ablating process is the above-mentioned debris screening [B. Majaron et al., ibid].

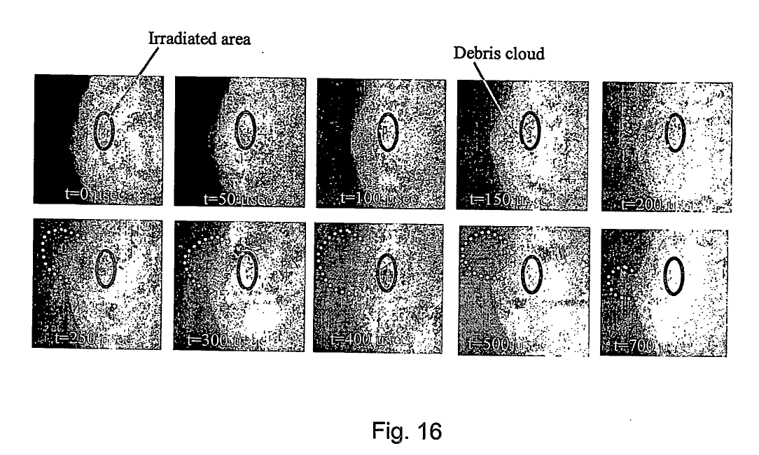

For a typical laser radiation in the infrared scale, there is no debris removal from the material during the first few tens of microseconds of the process. However, as time evolves, a debris cloud is formed and remains until about few hundreds of microseconds after the laser pulse ends (see, e.g., FIG. 16 in the Examples section). While the debris cloud exists, a substantial amount of the laser energy is absorbed by the debris cloud hence wasted.

According to a preferred embodiment of the present invention one or more of the scanning-parameters are selected so as to minimize debris screening. This may be done, for example, by scanning the material in such a manner that once or prior to the formation of the debris cloud (e.g., after 150 microseconds in the example shown in FIG. 16), the beam is diverted to another location where no debris cloud is in the light-path of the beam.

Another advantage of the scanning procedure is that this procedure may be exploited for ablating the material to form a predetermined geometrical surface. For example, when the ablation is done during a medical (e.g., dental) or any other procedure, the physician (or the operator) may select one or more of the scanning parameters so as to provide a predetermined ablation pattern. It is not intended to limit the scope of the invention to any specific ablation pattern. Thus, the predetermined ablation pattern may be a uniform pattern, a cylindrical pattern or any other regular or irregular pattern.

A particular feature of the present invention is that a predetermined amount of energy can be delivered to each location of the material. The predetermined amount of energy may be fixed for all the location or may vary from one location to the other, depending on the application for which the invention is used. For example, if the material strength is uniform and it is desired to obtain a uniform ablation pattern, then a fixed amount of energy is preferably delivered for all the location the material. For a material having harder regions and softer regions to which a uniform ablation pattern is to be applied, the amount of energy delivered to the harder regions is preferably higher than the amount of energy delivered to the softer regions. The amount of energy for each location may also be selected in accordance to the required ablation pattern, exploiting the proportion between the amount of absorbed energy and the depth of the ablation. One ordinarily skilled in the art would appreciate, however, that several non-uniformities may affect during the scanning procedure. These non-uniformities are preferably taken under consideration while selecting the appropriate scanning-parameters as will now be explained.

Hence, an ideal pulse for scanning would be such that carries a constant amount of energy, at any given time within the duration of the pulse. In other words, an ideal pulse would be a perfect square wave in the time-energy plane. Such pulse is, however, rarely attainable and in reality the pulse deviates from being square wave in particular at the beginning and the end of the pulse duration.

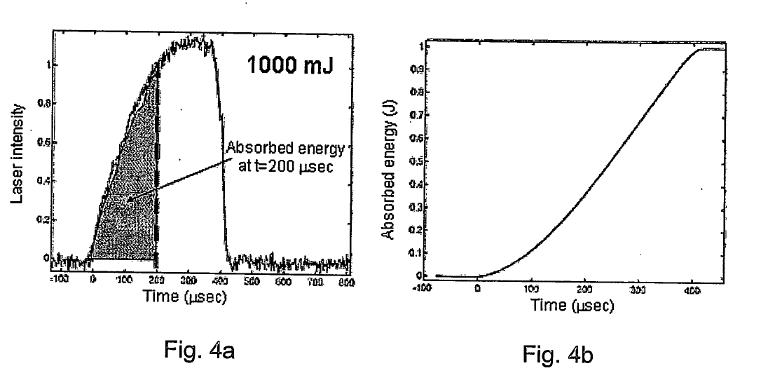

Reference is now made to FIGS. 4a-b, which are graphs that illustrate transient non-uniformities of intensity distribution within the pulse duration for a typical free running Er:YAG laser. FIG. 4a shows the shape of a 400 microseconds pulse in the time-intensity plane, and FIG. 4b shows the energy absorbed in the material as a function of time for the pulse duration of FIG. 4a. For a given time, t, the absorbed energy is the area bounded by the respective portion of intensity curve from the beginning of the pulse to time t. In FIG. 4a, an area corresponding to t=200 μsec is shaded for illustrative purposes.

As can be seen from FIGS. 4a-b, the pulse carries different amounts of energy at different times within its duration, and the absorbed energy graph is thus substantially non-linear.

According to a preferred embodiment of the present invention one or more scanning-parameters are selected so as to compensate transient non-uniformities of intensity distribution of laser radiation within duration of pulse. This may be done, e.g., by an appropriate modulation of the scanning-velocity using the intensity distribution (or a modification thereof) as a modulating function. For example, the scanning-velocity may be inversely proportional to the intensity distribution. Specifically, denoting the transient intensity of the laser by J(t), a preferred expression for the scanning-velocity is K/J(t), where K is a proportion constant. A typical value for K is from about 10³ J m/sec² to about 10⁴ J m/sec². Effects of the scanning-velocity and other parameters on the ablation process are further exemplified in the Examples section that follows.

Non-uniformity in the ablation process may also occur due to non-uniform spatial distribution of the laser intensity within the cross-sectional area of the beam. Such spatial non-uniformity may be compensated by the rotating beam about a longitudinal axis, in an angular velocity which is sufficiently high so that within the laser spot on the material, the delivered energy is substantially uniform. The beam can be rotated in any way known in the art, for example, using an optical element (e.g., a lens, a mirror, a prism, etc.) positioned in the light-path of beam and rotating the optical element about the longitudinal axis.

An additional factor which may be considered is the impinging angle of the beam on the material. The scanning procedure is preferably executed by dynamically diverting the beam, for example, using an arrangement of optical elements. Broadly speaking, there are two types of diversions which may be used. A first type, in which the impinging angle of the beam on the material is constant for all (or, at least, a majority) of the locations; and a second type in which there are different impinging angles at different locations. Both types of diversions are not excluded from the scope of the present invention and may be achieved in any method known in the art. For example, the first type of diversion may be achieved by deflecting the beam substantially parallel to itself, while the second type may be achieved by rotating the beam.

Different impinging angles at different locations of the material, however, may affect the laser flux and thereby the amount of energy delivered to each location. According to a preferred embodiment of the present invention one or more scanning-parameters are selected so as to compensate flux non-uniformities caused by different impinging angles of beam on different locations. This may be done, for example, by selecting the scanning-velocity to be small for large impinging angles and large for small impinging angles, were the impinging angles are measured relative to a normal to the material.

Referring now again to FIG. 2, according to a preferred embodiment of the present invention the method may further comprise an optional step, designated by Block 26, in which the material is cooled during the scanning process. The cooling may be done in any conventional way, such as, but not limited to, by a spray of liquid, e.g., water.

In another optional step of the method, designated by Block 28, at least one impinging-parameter of the beam on the material is continuously determined. The impinging-parameter is preferably an impinging-location or an impinging-angle. The impinging-parameter may be used for the purpose of ablation within predetermined boundaries, for example in medical application where, from safety reasons, tissues surrounding the ablated regions are not to be damaged. Thus, if one or more of the impinging parameters are in a predetermined risk range the laser radiation is preferably terminated.

According to a preferred embodiment of the present invention the impinging parameters may be determined by an additional laser beam, the wavelength of which is selected so as not to damage the surroundings of the material, as further detailed hereinafter.

Thus, the method, according to the present aspect of the invention successfully provides solutions to the various problems associated with ablation, in general and ablation of hard material in particular. As stated the material can be any material which is sufficiently responsive to laser radiation to allow ablation therewith. More specifically, the material may be a hard tissue of a mammal, hence, the method may be used in many medical procedures, such as, but not limited to, dental procedure (e.g., crown preparation, dental implantation, caries removal, endodontic treatment, enamel and dentin preparation and conditioning), bones surgery (e.g., bone tumor treatments, bone transplantation and the like), orthopedic procedures and the like.

The present invention successfully provides an apparatus and a system which may be used for executing one or more of the above method steps.

According to another aspect of the present invention there is provided an apparatus 50, for scanning a material by a beam of laser radiation. The laser radiation is in a form of plurality of pulses, as further detailed hereinabove.

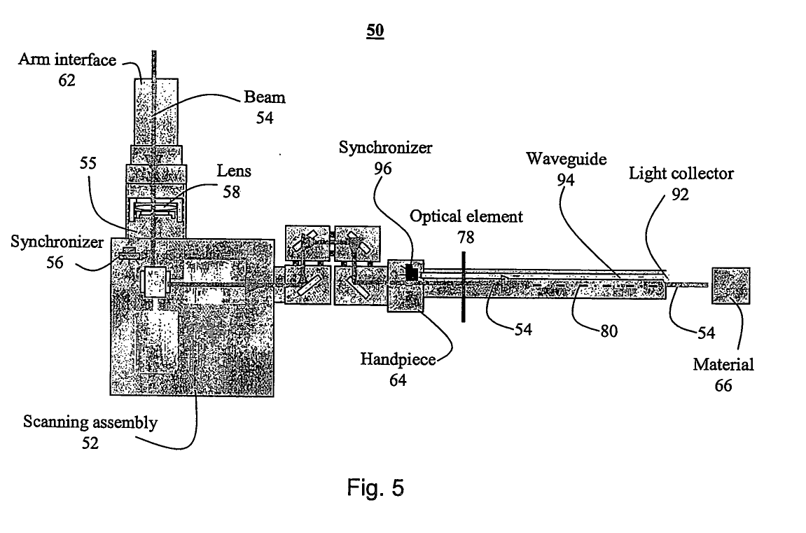

Reference is now made to FIG. 5, which is a schematic illustration of apparatus 50. Apparatus 50 comprises a scanning assembly 52 for dynamically diverting beam 54, within a duration of a pulse, so as to transfer a predetermined amount of energy to each location of the material as further detailed hereinabove.

According to a preferred embodiment of the present invention apparatus 50 may further comprise a synchronizer 56 for synchronizing scanning assembly 52 and a laser device (not shown in FIG. 5) which generates the beam. Any synchronizer known in the art may be used, such as, but not limited to, an optical synchronizer or an electrical synchronizer. Synchronizer 56 which is shown in FIG. 5 is an optical synchronizer, which may operate as follows. A lens 58, which may also be used as a focusing lens, is positioned in the light-path of beam 54. A fraction 55 of the laser radiation is scattered off lens 58 and reaches synchronizer 56, while beam 54 continues towards scanning assembly 52. Being in communication with scanning assembly 52, synchronizer 56 receives information from beam 54 and transmits the information in real time to scanning assembly 52. Many optical synchronizers exist and may be used as synchronizer 56, one such optical synchronizer is a Mercury Cadmium Telloride PhotoVoltaic (MCT PV) detector.

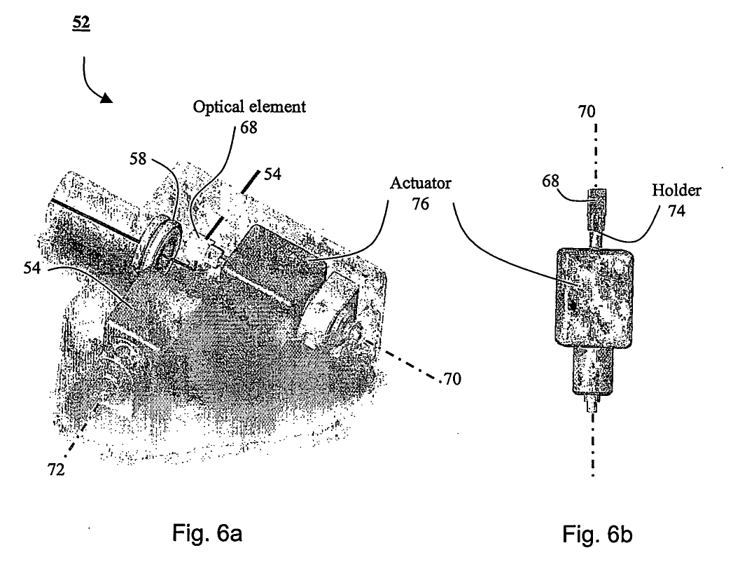

Scanning assembly 52 is better illustrated in FIGS. 6a-b. According to a preferred embodiment of the present invention, scanning assembly 52 comprises one or more optical elements 68 positioned in the light-path of beam 54. Optical element(s) 68 may be, for example, a lens, a mirror, a prism or a combination thereof.

Each one of optical elements 68 preferably connected via a holder 74 to an actuator 76 (e.g., a galvanometric actuator) which rotates about axis 70 or axis 72 so that beam 54 is dynamically diverted.

One ordinarily skilled in the art would appreciate that other arrangements of optical elements may be used for the purpose of diverting beam 54 either by rotation about axes 70 and 72 or by deflecting beam 54 substantially parallel to itself as further detailed hereinabove. Irrespectively of the arrangements of optical elements, scanning assembly preferably generates one- two- or three-dimensional scanning of material 66.

Referring now again to FIG. 5, apparatus 50 may further comprise one or more optical elements 78 positioned in a light-path of the beam and serves for rotating beam 54 about longitudinal axis 80, so as to compensate the above-motioned spatial non-uniformities of intensity distribution. Optical element 78 may be, for example a passive beam homogenizer, which is known per se, and the like.

According to a preferred embodiment of the present invention, apparatus 50 may further comprise an arm interface 62 for mounting scanning assembly 52 to an articulated arm (not shown in FIG. 5). Additionally, apparatus 50 may further comprise a hingedly attached handpiece 64 so that the operator can easily grip apparatus 50 and rotate handpiece 64 to one of several open positions so as to better direct beam 54 to material 66.

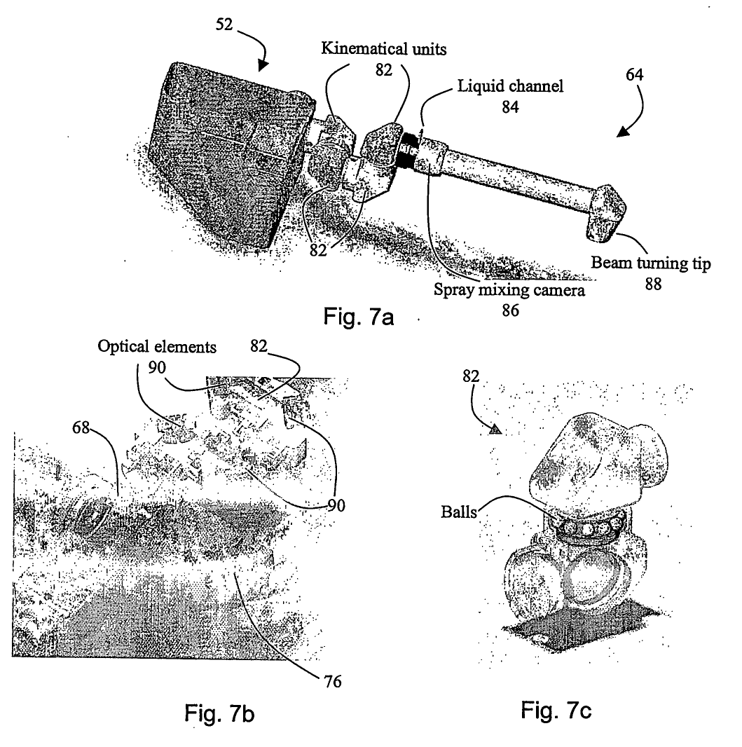

Handpiece 64 is better illustrated in FIGS. 7a-c. Referring to FIG. 7a, handpiece 64 preferably comprises a plurality of kinematical units 82 which provide the required degrees-of-freedom for the rotation of handpiece 64. Handpiece may further comprise one ore more liquid channel 84, for providing liquid to material 66 while scanning, so as to cool the material as further detailed hereinabove. Several liquid channel may be used, one for each liquid. For example, one liquid channel may be used for water and another for air. Other combinations of liquids are also not excluded (e.g., liquids in different temperature and the like). Additionally handpiece may further comprise a spray mixing camera 86 and/or a beam turning tip, positioned at the end of handpiece 64 for an additional turning of beam 54 prior to the impingement on material 66. Spray mixing camera 86 serves for creating the liquid spray by combining jets of, e.g., water and air.

The interior of the portion of handpiece 64 which include kinematical units 82 is shown in FIG. 7b. Also shown in FIG. 7b is the relative location of kinematical units 82 optical elements 68 and actuators 76. Hence, kinematical units 82 include a plurality of optical elements 90 (e.g., mirrors) which are designed so as to direct beam 54 through handpiece 86. Other means for directing the beam through handpiece 86 (e.g., optical fibers) are not excluded. FIG. 7c is an enlarged view of kinematical unit 82, which preferably comprises a plurality of small balls (typically about 3 mm in diameter) which facilitate the rotation of kinematical unit 82.

Apparatus 50 may also be designed and constructed for determining impinging-parameter of beam 54 on material 66. As stated, this is preferably done by an additional laser beam.

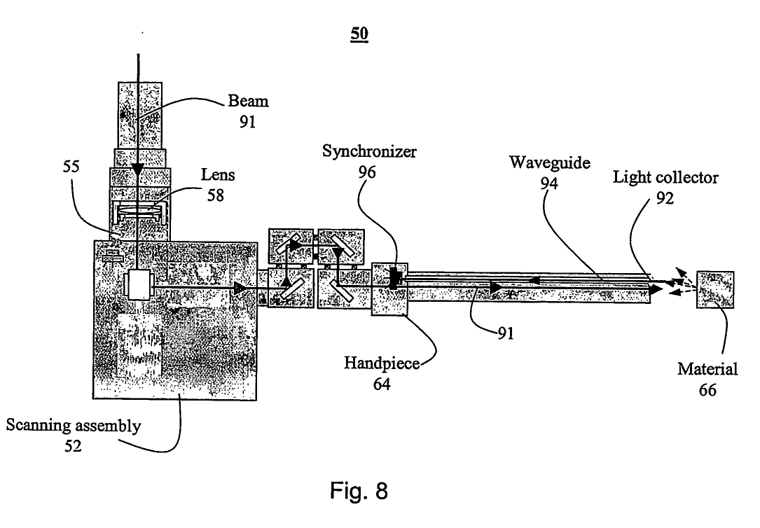

Reference is now made to FIG. 8, which is a simplified illustration of the light-path of additional laser beam 91 within apparatus 50. According to a preferred embodiment of the present invention apparatus 50 may comprise a light collector 92 for collecting beam 91 when beam 91 is reflected from material 66. In addition, apparatus 50 preferably comprises a waveguide 94 and an additional synchronizer 96 communicating with the laser device which generates beam 54. Waveguide 94 serves for directing beam 91 to synchronizer 96, and synchronizer 96 serves for synchronizing the laser device and beam 91. Synchronizer 96 may be, for example, a photodiode which generate a signal once impinged by beam 91. This signal may be used for terminating the primary laser beam (i.e., beam 54), once the signal enters a predetermined risk range.

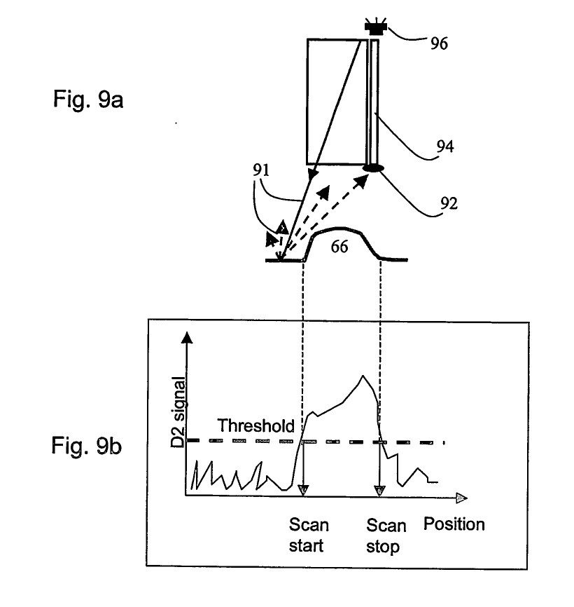

One embodiment of the procedure of terminating and reactivating beam 54 may be better understood from FIGS. 9a-b. FIG. 9a shows the surface of material 66 and a portion of beam 91 and FIG. 9b shows the respective signals received from synchronizer 96. Hence, if beam 91 impinges outside material 66 the distance between light collector 92 and the impinged surface is large, so that by the time the reflected beam 91 reaches synchronizer 96 most of the energy of beam 91 has been already scattered off, and the signal synchronizer 96 is small. In this case, the primary laser beam is terminated. On the other hand, when beam 91 impinges on material 66 the signal which is generated in synchronizer 96 is high and the scanning continues (or resumed).

Laser beam 91 is preferably characterized by a wavelength which does not damage material 66 or its surroundings. For example, if the present embodiment is used for a dental procedure, the wavelength of laser beam 91 is preferably selected so as not to damage the soft tissue surrounding the ablated tooth. According to a preferred embodiment of the present invention laser beam 91 is generated by an additional laser device. Alternatively, a combined laser device, which is capable of generating both beam 54 (which ablates material 66) and beam 91 (which is used solely for tracking purposes) may be used. For hard tissue applications, the wavelength of beam 91 is preferably from about 0.4 μm to about 1.1 μm.

According to an additional aspect of the present invention, there is provided a system for ablating a material, generally referred to herein as system 100.

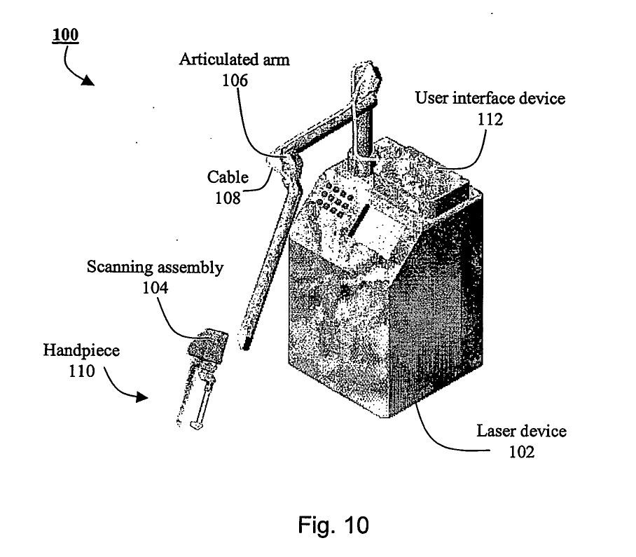

Reference is now made to FIG. 10, which is a schematic illustration of system 100. In its basic configuration, system 100 comprises a laser device 102 and a scanning assembly 104. Laser device serves for generating a beam of laser radiation in a form of plurality of pulses, e.g., beam 54. The principles and operations of scanning assembly 104 are similar to principles and operations of scanning assembly 52 as further detailed hereinabove with respect to apparatus 50.

According to a preferred embodiment of the present invention system 100 may further comprise an articulated arm 106 (or a plurality of articulated arms, if more than one arm is required) onto which scanning assembly 104 is mounted. Preferably, the laser radiation from laser device 102 is guided through arm 106, e.g., using a fiber-optic cable 108 or any other components which is capable of guiding a beam of laser. Arm 106 may be any known articulated arm such as, but not limited to, the articulated arms which may be found in dentistry clinics.

System 100 may also comprise a handpiece 110, which may be similar to handpiece 64, as further detailed hereinabove.

According to a preferred embodiment of the present invention system 100 may further comprise a user interface device 112 electrically communicating with scanning assembly 104. User interface device 112 serve for receiving the scanning-parameters from the operator and transmitting the scanning-parameters to scanning assembly 104.

According to still another aspect of the present invention, there is provided a method of crowning a tooth. The method can be performed in a dentistry clinic, in veterinary clinic or in any other location (outdoors or indoors), for treating humans and/or other animals, such as, but not limited to mammals. The method comprises the following method steps which are illustrated in the flowchart of FIG. 11.

Referring to FIG. 11, in a first step of the method, designated by block 122, a beam of laser radiation is generated, similarly to beam 54. In a second step, designated by Block 124, the tooth is scanned within a duration of a pulse, as further detailed hereinabove. In a third step, designated by Block 126, the second step is repeated a number of times which is required to ablate an external surface of the tooth. More specifically, the scanning is continued until the surface of the tooth is sufficiently small so that a crown can be positioned on the tooth without interfering to adjacent teeth. In a fourth step of the method, designated by Block 128 in FIG. 11, a crown, compatible to the surface of the tooth, is provided and positioned onto the tooth.

In all the embodiments above, the primary beam may be generated by any a laser device capable of providing laser radiation which ablate the material to some extent. These include, but are not limited to, the following laser devices: Er based laser device, Ho:YAG laser device, carbon-dioxide laser device, Nd based laser device and laser diode device. Er based laser devices include, but are not limited to, Er:YAG, Er:YSGG, Er:glass and the like. Nd based laser devices include, but are not limited to, Nd:YAG, Nd:YLF, Nd:glass and the like. In addition, according to a preferred embodiment of the present invention the device generates polarized radiation so as to optimize the efficiency.

It is expected that during the life of this patent many relevant devices for generating ablative laser radiations will be developed and the scope of the term laser radiation in this context is intended to include all such new technologies a priori.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

### Example 1

### A Model for Laser-Tissue Interaction

Theory

A theoretical model for laser-tissue interaction dynamics has been developed. The model takes into account the non-linearity of the laser absorption coefficient and the non-uniform intensity distribution within the laser pulse.

The non-linearity range of the absorption coefficient is known to be significant for extremely high applied energies, such as ablation energies.

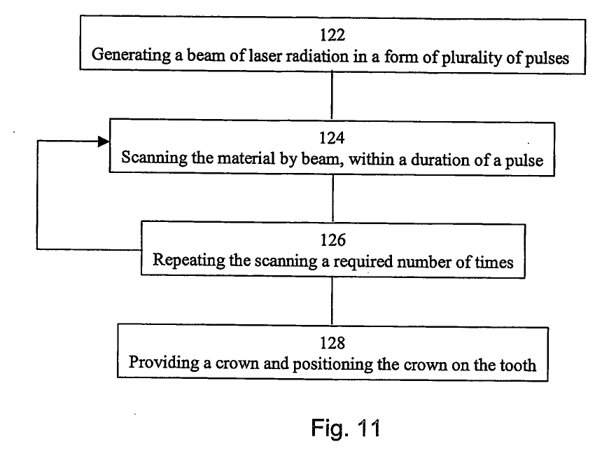

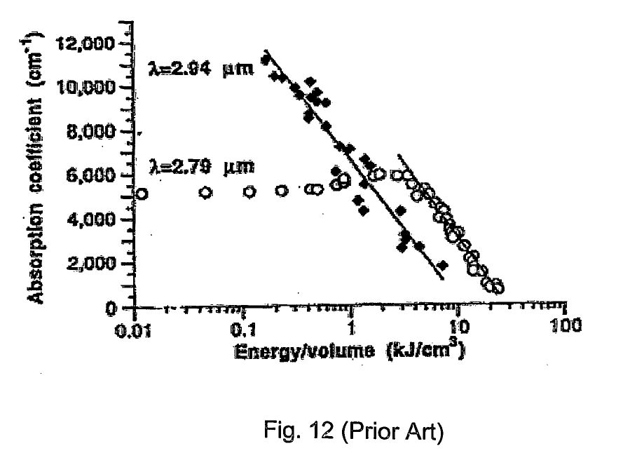

FIG. 12 shows results of measurements of absorption coefficient, α, of water as a function of the applied energy density, were the absorption coefficient, α, is presented on a linear scale in units of cm⁻¹ and the energy density is presented on a logarithmic scale in units of J/cm³ [A. Saar, D. Gal, R. Wallach, S. Akselrod, A. Katzir, Appl. Phys. Lett 50, 1556 (1987)]. The non-linearity of the absorption coefficient is vivid.

Generally, the experimental results, presented in FIG. 12, may be parameterized using the following equation, defined for three different energy regions;

(i) a low-energy region, for energies, E, which are below a threshold energy, E_{threshold}, (ii) an intermediate-energy region, for energies which are between the threshold energy and a saturation energy, Eₛₐₜᵤᵣₐₜᵢₒₙ; and (iii) a high-energy region, for energies which are above the saturation energy:

$$α ( t , z ) = \{ \begin{matrix}{α}_{0} & E ( t , z ) < {E}_{threshold} \\ A - γ {log}_{10} ( \frac{E ( t , z )}{{E}_{threshold}} ) & {E}_{threshold} < E ( t , z ) < {E}_{saturation} \\ {α}_{∞} & E ( t , z ) > {E}_{saturation} ,\end{matrix} \qquad EQ . 1$$

where t is the time and z is the penetration depth of the energy into the material. According to the parameterization of Equation 1, in the low-energy region, the absorption coefficient is a constant, α₀, in the intermediate-energy region the absorption coefficient is a logarithmically decreasing function of the applied energy, and in the high-energy region the absorption coefficient saturates to a constant, α_{∞}, lower than, α₀. The third region ensures that α remains positive at all energies, as it should, from first principles (the gain possibility is neglected). The saturation of the absorption coefficient is explained by another absorption processes which becomes dominant when α is small.

The values of the parameters of Equation 1 depend on the material. For water, the parameters are given in Equation 2, below:
E_{theshold}=0.1 kJ/cm³
Eₛₐₜᵤᵣₐₜᵢₒₙ=12.3 kJ/cm³
A=1.15 10⁻² μm⁻¹
γ=5.5 10⁻³ μm⁻¹
α₀=1.15 10⁻³ μm⁻¹
α_{∞}=10⁻⁴ μm⁻¹. EQ. 2

The model calculates the absorbed amount of energy within the tissue during a laser pulse generated by an Er:YAG, taking into account typical transient non-uniformities of intensity distribution (in this respect, see FIGS. 4a-b above) thereof. Denoting intensity of the laser impinging the material's surface by J(t), and the laser the laser flux by F(z, t), the time-dependence of the laser flux is expressed by the following equation:

$$\frac{\partial F ( t , 0 )}{\partial t} = J ( t ) , \qquad EQ . 3$$

Spatial non-uniformity of the laser beam is presently neglected. While propagating through the material, the laser intensity decreases due to the tissue-laser interactions, which are assumed to be dominated by absorption. The change of flux, as a function of the penetration depth, z, can be written as:

$$\frac{\partial F ( t , z )}{\partial z} = α ( t , z ) F ( t , z ) \qquad EQ . 4$$

The relation between the laser flux, F, and the total amount of absorbed energy, E, is:

$$E ( t , z ) = {\int }_{{t}^{′} = 0}^{{t}^{′} = t} F ( {t}^{′} , z ) ⅆ {t}^{′} \qquad EQ . 5$$

Thus, the absorption coefficient, α, depends on the energy, E, through Equations 3-5.

Numerical Calculations

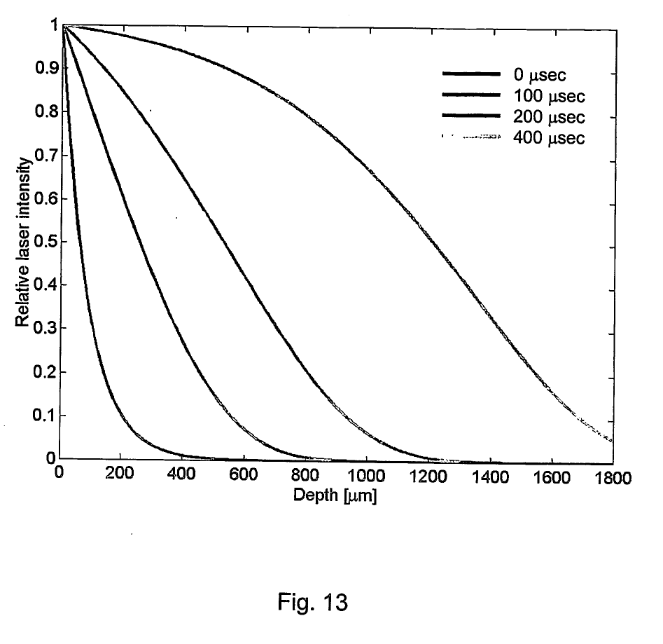

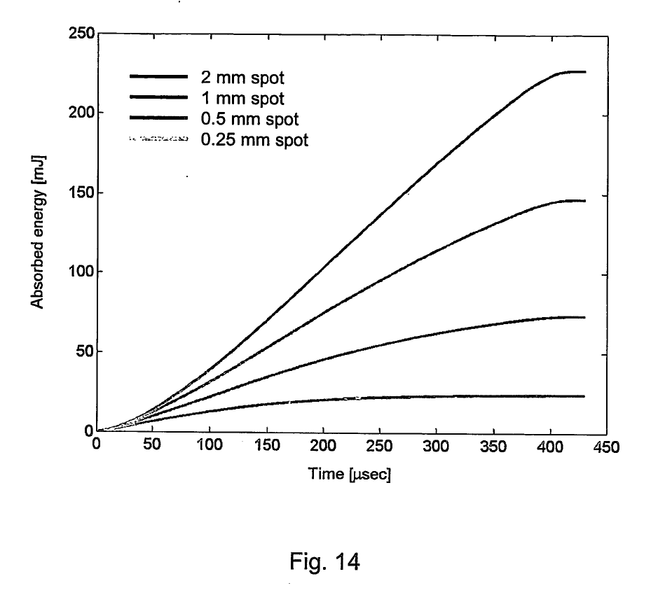

The absorption equation (Equation 4) was solved numerically by a finite differences technique, for various initial conditions. The results of the calculations are presented, below with reference to FIG. 13-14.

FIG. 13 shows results of depth profiles of the laser intensity distribution at various times during a laser pulse having a total energy of 1000 mJ per pulse and a laser spot diameter of 0.3 mm, at the following times 0 μm, 100 μm, 200 μm and 400 μm.

Initially (at t=0) 25% of the laser energy is absorbed within an upper layer of 150 μm. As the time evolves the amount of absorbed energy within the tissue increases and consequently the absorption coefficient decreases. This causes to an increase in the laser penetration depth. For example, absorption of 25% of the laser energy penetrates through 400 μm of tissue at t=100 μm, 800 μm of tissue at t=200 μm, and 1500 μm of tissue at t=400 μm (the end of the pulse duration).

FIG. 14 shows the total amount of the absorbed energy within the top 40 μm of tissue for a pulse of 1000 mJ applied to spot sizes of 0.25 mm, 0.5 mm, 1 mm and 2 mm. For 2 mm spot the absorbed energy grows almost linearly with time, while for smaller spot sizes the rate of change of the absorbed decreases. For spot size of 0.25 mm, 90% of the energy is deposited within the first 100 μsec, and to a good approximation remain constant for t>100 μsec. In other words, the energy penetrates deeper into the tissue without contribution to the ablation process. The same effect occurs for larger pulse energy.

Conclusions

The above calculations demonstrate that for a 0.25 mm spot, the energy of the laser radiation may be distributed efficiently by treating 4 spots within a duration of a single pulse. This can be achieved by the scanning procedure as further detailed hereinabove.

### Example 2

### Experimental Investigations of Hard Tissue Ablation

Methods

An Er:YAG laser was used for ablating hard tissues of freshly extracted human teeth. The goal of the experiments was to study the dynamic of the interaction between a hard tissue and a laser beam.

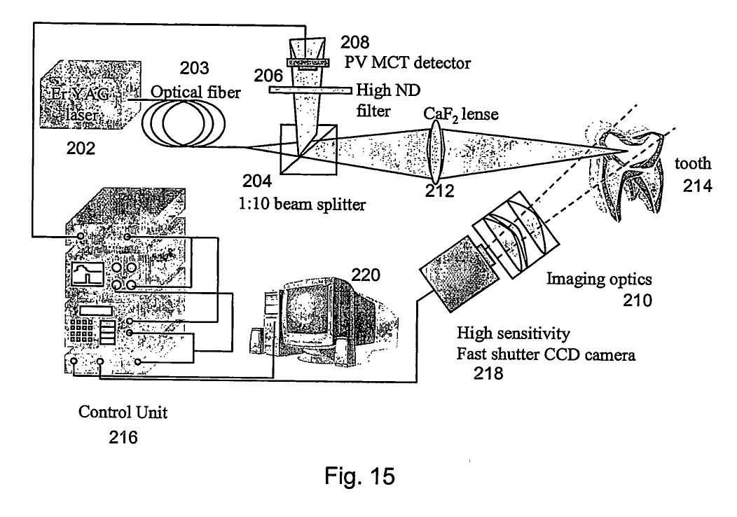

The experimental system is schematically shown in FIG. 15.

A beam of laser emitted from an Er:YAG laser 203 was guided by an optical waveguide 203 to a beam splitter 204. Beam splitter 204 directed about 90% of the beam to a CaF₂ lens 212 which focused the beam onto tooth 214, while the remaining 10% of the beam was directed through a High ND filter 206 to a detector 208 (photovoltaic Mercury-Cadmium-Telluride) and was used for synchronization. The synchronization was governed by a control unit 216, and a computer 220 was used for collecting data. A sensitive fast CCD camera 218, synchronized with the laser beam was used, together with an arrangement 210 of imaging optical elements for imaging tooth 214. Control unit 216 included a 1 MHz bandwidth detector amplifier for amplifying the signals received from detector 208, a digital delay generator for generating an appropriate delay of the signal, an oscilloscope and a camera controller for transmitting signals to the shutter of camera 218.

Tooth 214 was ablated by laser 203 using the following parameters: wavelength of 2.94 μm, energy of 700 mJ per pulse and pulse duration of 400 μsec.

Results

Reference is now made to FIG. 16, which is a series of 10 images of tooth 214 taken by camera 218, at times 0, 50, 100, 150, 200, 250, 300, 400, 500 and 700 μsec from the beginning of a representative pulse. Shown in the images are the irradiated area (red) and debris cloud (orange) during the radiation. As can be seen from FIG. 16, there is no debris removal from the material during the first 150 μsec of the process. However, after 150 μsec of radiation, a debris cloud is formed and remains until about 300 μsec after the laser pulse ends. While the debris cloud exists, a substantial amount of the laser energy is absorbed by the debris cloud hence wasted.

### Example 3

### Fast Scanning of Hard Tissues

Methods

An Er:YAG laser of Example 2, was used for ablating hard tissues of freshly extracted human teeth, employing features of the method of the present invention. The goal of the experiments was to optimize the scanning-parameters and to study the effect thereof on the efficiency and quality of the ablating process.

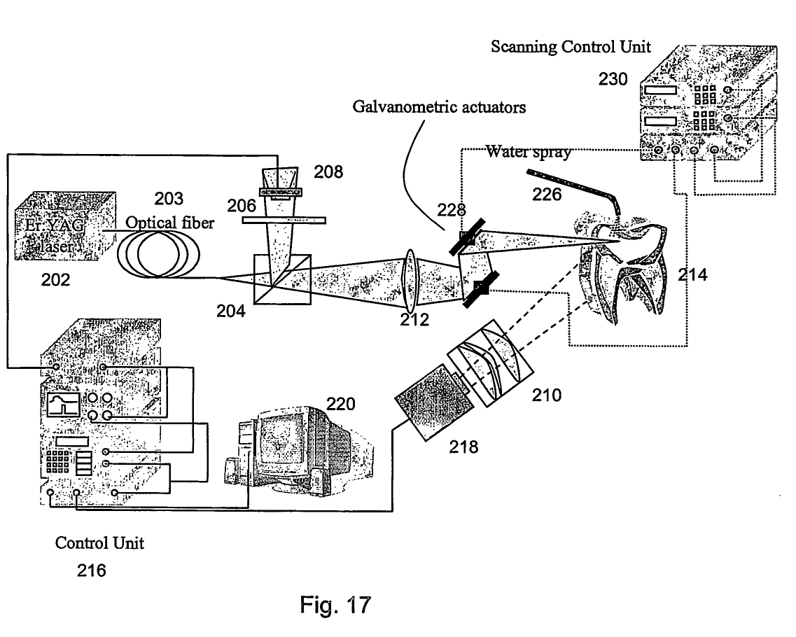

The experimental system is schematically shown in FIG. 17. The laser radiation and the synchronization with camera 218 were as further detailed hereinabove in Example 2.

A scanning assembly, essentially as detailed hereinabove was used for scanning tooth 214 with the laser beam. Two galvanometric actuators 228 were used for dynamically diverting the beam.

A polished gold mirror 8×8 cm in lateral dimension and 1 mm in thickness was manufactured and integrated on galvanometric actuators 228 so as to achieve a minimal moment of inertia. The resulting bandwidth of the scanning assembly was 1.2 kHz. A scanning control unit 230 was provided the required synchronization for the galvanometric actuators.

Following are descriptions of four experiments, performed by the Inventors of the present invention, using the experimental system of FIG. 17.

Experiment 1: The Effect of Scanning-Frequency

The effect of the scanning-frequency was investigated by irradiating enamel from freshly extracted human tooth by a scanned laser beam using different one-dimensional scanning-frequencies.

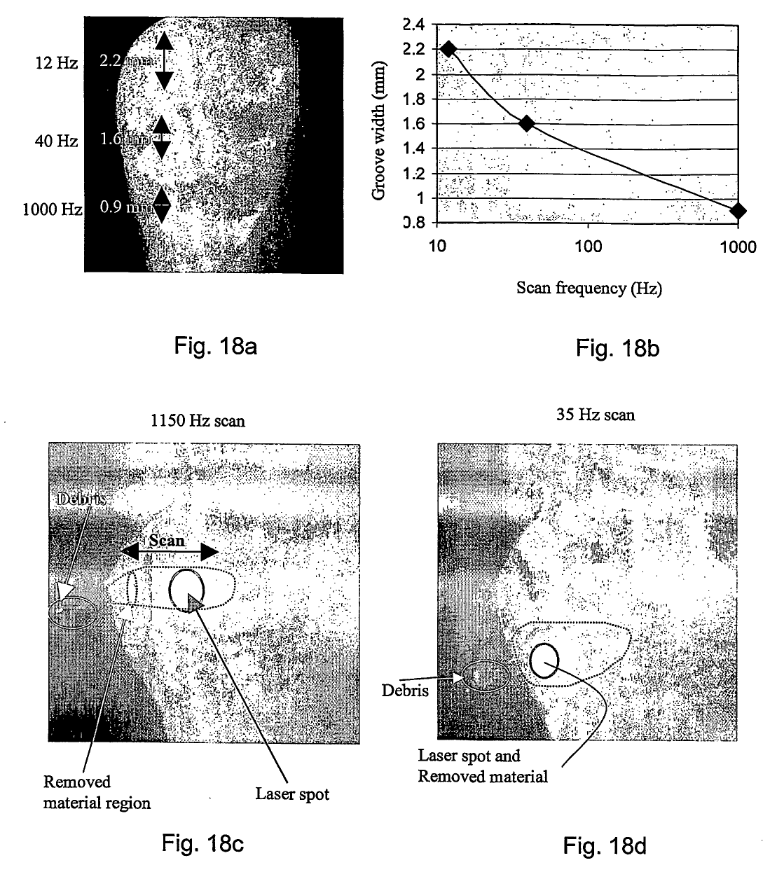

FIG. 18a is an image of the enamel for different scanning-frequencies. Each scanning-frequency resultant in a formation of a groove of a different width in the enamel. Specifically, a 12 Hz scanning formed a 2.2 mm groove, a 40 Hz scanning formed a 1.6 mm groove and a 1000 Hz scanning formed a 0.9 mm groove.

FIG. 18b is a graph showing the width of the formed groove as a function of the scanning-frequency.

The effect of high scanning-frequency can be better understood from FIGS. 18c-d, which are images taken by camera 218 while scanning the enamel with scanning-frequencies of 1150 Hz (FIG. 18c) and 35 Hz (FIG. 18d). As can be seen From FIG. 18c, for the scanning-frequency of 1150 Hz, the laser spot is in a substantially remote location relative to the removed material region hence is not affected by the debris cloud. On the other hand, for the scanning-frequency of 35 Hz (FIG. 18d), the laser spot and the removed material region overlap and therefore the debris cloud screens the laser radiation hence reduces the efficiency.

Experiment 2: Modulation of the Scanning-Velocity

As stated, the laser pulse typically deviates from being square wave, in particular at the beginning and the end of the pulse duration (see FIG. 4a). This experiment was directed at (i) studying the effect of transient non-uniformities of intensity distribution on the ablation process; and (ii) modulating the scanning-velocity so as to compensate this effect.

For the purpose of studying the effect of transient non-uniformities on the ablation process, a constant scanning-velocity was used.

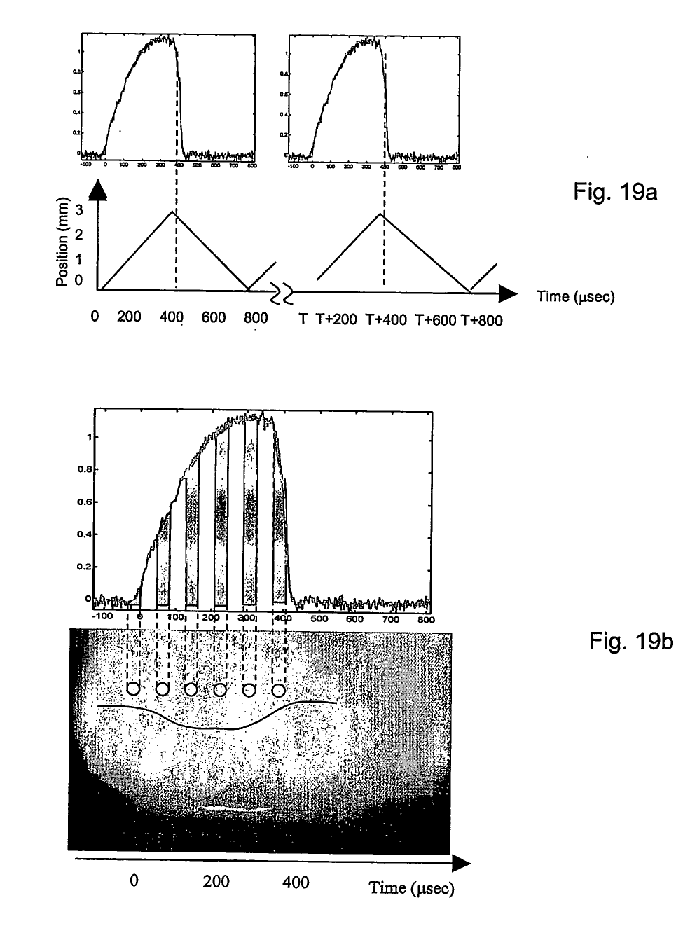

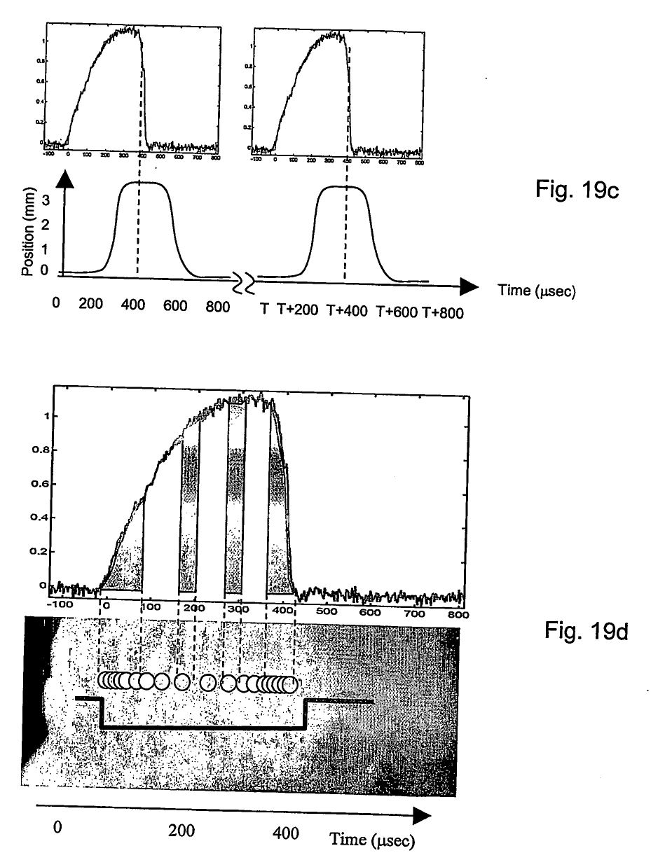

FIG. 19a shows the pulse shape and the position of the laser spot as a function of time within the duration of the pulse, for constant scanning-velocity. Equally spaced time-intervals thus correspond to equally spaced positions of the laser spot.

FIG. 19b illustrates the amount of energy delivered to each location on the enamel sample, when the constant scanning-velocity was used. As can be seen from FIG. 19b, the constant scanning-velocity resultant in a non-uniform ablation depth, because different amount of energy was delivered to different locations.

FIG. 19c show the pulse shape and a profile of the modulated scanning-velocity which was used for compensating the effect of transient non-uniformities.

FIG. 19d, shows several positions of the laser spot on the enamel, when the modulated scanning-velocity was employed. As can be seen from FIG. 19d, a crater was formed with a precise and uniform depth. Thus, the modulation of the scanning-velocity substantially reduced the above effect.

Experiment 3: Ablating Large Area of Enamel

An enamel layer of a human tooth was irradiated by laser 203 for a period of 90 seconds. During the experiment, water spray 226 was constantly used for cooling the sample. The laser and scanning parameters were as follows: energy of 600 mJ per pulse, pulse repetition rate of 12 pulses per second, pulse duration of 390 μsec, horizontal scanning-frequency of 1200 Hz, vertical scanning-frequency of 50 Hz and a modulated scanning-velocity.

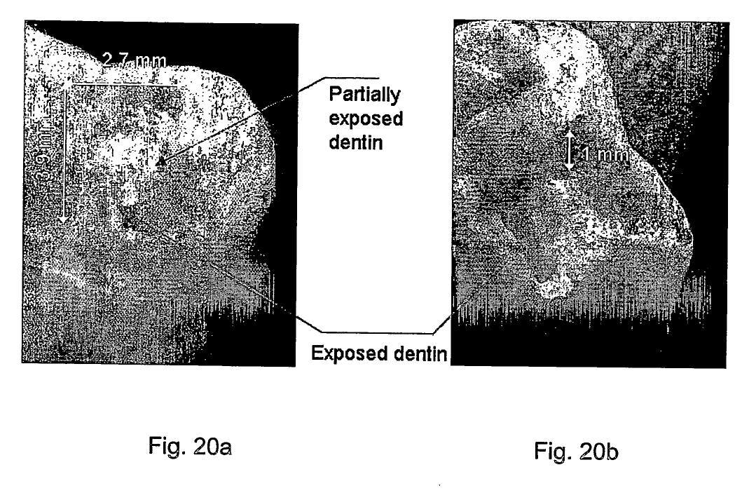

FIGS. 20a-b are images of the enamel after the 90 seconds ablation procedure. A large volume of enamel has been successfully ablated, forming a crater with precise predetermined dimensions of 2.7 mm×3.9 mm×1.0 mm. As can be seen from FIG. 20, the walls of the formed crater are substantially smooth.

Experiment 4: Ablating Large Area of Dentin

A dentin layer of a human tooth was irradiated by laser 203 for a period of 30 seconds. During the experiment, water spray 226 was constantly used for cooling the sample. The laser and scanning parameters were: energy of 600 mJ per pulse, pulse repetition rate of 12 pulses per second, pulse duration of 390 μsec, horizontal scanning-frequency of 1200 Hz, vertical scanning-frequency of 40 Hz and a modulated scanning-velocity.

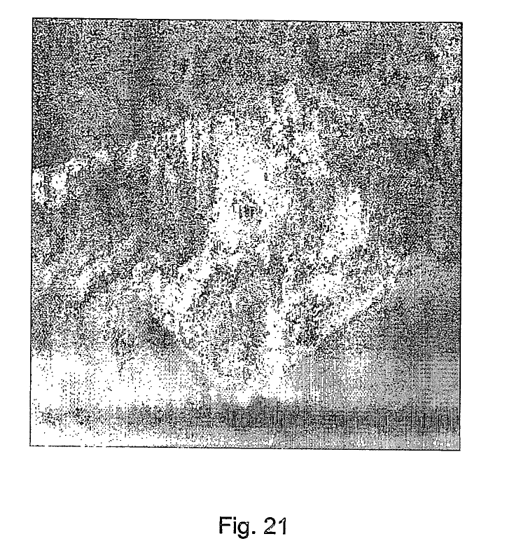

FIG. 21 is an image of the dentine after the 30 seconds ablation procedure. The dimensions of formed crater were 3.4 mm×5.7 mm×0.8 mm. In this experiment, a non-uniform scanning waveform was used so that many different tissue depths were achieved during a single procedure. As in the enamel experiment, the walls of the crater formed in the dentin are substantially smooth, and the dimensions of the crater were achieved to a substantially high precision.

Experiment 5: Ablating Large Area of Bone Tissue

Large area removal of bone tissue may be employed, for example, in dental implantations, where a precise holes is required in the bone. In this experiment, a facial bone taken from bovine was irradiated by laser 203 for a period of 30 seconds.

The following laser parameters were used: energy of 600 mJ per pulse, pulse repetition rate of 12 pulses per second, pulse duration of 390 μsec, horizontal scanning-frequency of 1100 Hz, vertical scanning-frequency of 45 Hz and a modulated scanning-velocity.

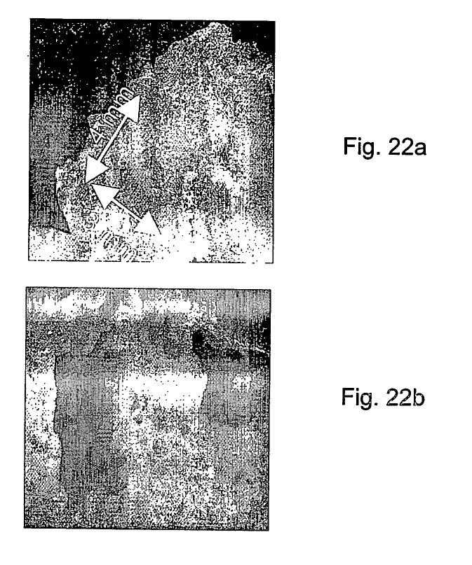

FIGS. 22a-b are images of the bone tissue after the 30 seconds ablation procedure. The bone tissue was successfully and accurately removed. The dimensions of the formed crater were 3 mm×4 mm×6.5 mm.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1-202. (canceled)

203. A method of ablating a material, the method comprising:
(a) generating a beam of laser radiation in a form of plurality of pulses, said laser radiation having a wavelength suitable for ablating the material; and
(b) within a duration of a pulse of said plurality of pulses, scanning the material by said beam, so as to transfer a predetermined amount of energy to each one of a plurality of locations of the material, said predetermined amount of energy being selected so as to ablate the material.

204. The method of claim 203, wherein said scanning is characterized by at least one scanning-parameter, said at least one scanning-parameter is selected from the group consisting of a scanning-frequency, a scanning-velocity, a scanning-acceleration, a scanning-amplitude, a scanning-angle, a scanning-pattern and a scanning-duration.

205. The method of claim 204, wherein said at least one scanning-parameter is selected so as to compensate spatial non-uniformities of intensity distribution of said laser radiation.

206. The method of claim 204, wherein said at least one scanning-parameter is selected so as to compensate transient non-uniformities of intensity distribution of said laser radiation within said duration of said pulse.

207. The method of claim 204, wherein said at least one scanning-parameter is selected so as to compensate flux non-uniformities caused by different impinging angles of said beam on said plurality of locations of the material.

208. The method of claim 204, wherein said compensating said flux non-uniformities is by selecting said scanning-velocity to be small for large impinging angles and large for small impinging angles, said large impinging angles and said small impinging angles being measured relative to an imaginary line positioned normal to the material.

209. The method of claim 204, wherein said scanning is by dynamically diverting said beam, so as to provide a substantially constant impinging angle of said beam on each of said plurality of locations of the material.

210. The method of claim 203, further comprising cooling the material during said scanning.

211. An apparatus for scanning a material by a beam of laser radiation being in a form of plurality of pulses, the apparatus comprising a scanning assembly for dynamically diverting the beam, within a duration of a pulse of the plurality of pulses, so as to transfer a predetermined amount of energy to each one of a plurality of locations of the material, thereby to scan the material by the beam.

212. The apparatus of claim 211, further comprising a synchronizer for synchronizing said scanning assembly and a laser device generating the beam.

213. The apparatus of claim 211, wherein said scanning assembly comprises at least one optical element positioned in a light-path of the beam, said at least one optical element being operable to rotate thereby to dynamically divert the beam.

214. The apparatus of claim 212, wherein said scanning assembly is operable to preserve a substantially constant impinging angle of the beam on each of said plurality of locations of the material.

215. The apparatus of claim 211, wherein said scanning assembly is designed and constructed to scan the material in such a manner that spatial non-uniformities of intensity distribution of the laser radiation are compensated.

216. The apparatus of claim 211, wherein said scanning assembly is designed and constructed to scan the material in such a manner that transient non-uniformities of intensity distribution of the laser radiation within said duration of said pulse are compensated.

217. The apparatus of claim 211, wherein said scanning assembly is designed and constructed to scan the material in such a manner that flux non-uniformities, caused by different impinging angles of the beam on said plurality of locations of the material, are compensated.

218. The apparatus of claim 211, wherein said scanning assembly is operable to provide a small scanning-velocity for large impinging angles and a large scanning-velocity for small impinging angles, thereby to compensate said flux non-uniformities, said large impinging angles and said small impinging angles being measured relative to a normal to the material.

219. The apparatus of claim 212, further comprising a light collector for collecting said additional laser beam when said additional laser beam is reflected from the material, thereby to determine at least one impinging-parameter of said beam on the material.

220. A system for ablating a material, the system comprising:
(a) a laser device for generating a beam of laser radiation in a form of plurality of pulses, said laser radiation having a wavelength suitable for ablating the material; and
(b) a scanning assembly, electrically communicating with said laser device, said scanning assembly being capable of scanning the material by said beam, within a duration of a pulse of said plurality of pulses, so as to transfer a predetermined amount of energy to each one of a plurality of locations of the material, said predetermined amount of energy being selected so as to ablate the material.

221. The system of claim 220, wherein said scanning assembly comprises a synchronizer for synchronizing said scanning assembly and said laser device.

222. The system of claim 221, wherein said synchronizer is selected from the group consisting of an optical synchronizer and an electrical synchronizer.

223. The system of claim 220, wherein said scanning assembly is operable to dynamically divert said beam thereby to scan the material by the beam.

224. The system of claim 223, wherein said scanning assembly comprises at least one optical element positioned in a light-path of said beam, said at least one optical element being operable to rotate thereby to dynamically divert said beam.

225. The system of claim 223, wherein said scanning assembly is operable to preserve a substantially constant impinging angle of the beam on each of said plurality of locations of the material.

226. The system of claim 220, wherein said scanning assembly is designed and constructed to scan the material in such a manner that spatial non-uniformities of intensity distribution of said laser radiation are compensated.

227. The system of claim 220, wherein said scanning assembly is designed and constructed to scan the material in such a manner that transient non-uniformities of intensity distribution of said laser radiation within said duration of said pulse are compensated.

228. The system of claim 227, wherein said scanning assembly is operable to provide a scanning-velocity which is inversely proportional to said intensity distribution, thereby to compensate said transient non-uniformities of said intensity distribution.

229. The system of claim 220, wherein said scanning assembly is designed and constructed to scan the material in such a manner that flux non-uniformities, caused by different impinging angles of said beam on said plurality of locations of the material, are compensated.

230. The system of claim 220, wherein said scanning assembly is operable to provide a small scanning-velocity for large impinging angles and a large scanning-velocity for small impinging angles, thereby to compensate said flux non-uniformities, said large impinging angles and said small impinging angles being measured relative to a normal to the material.

231. The system of claim 220, further comprising a cooling apparatus.

232. The system of claim 221, further comprising an additional laser device for generating an additional laser beam.
